(19) United States
(12) Patent Application Publication
(10) Pub. No.: US 2003/0170662 A1
(43) Pub. Date: Sep. 11, 2003

(54) Method for screening substance specifically inhibiting non-mevalonate pathway

(76) Inventors: Haruo Seto, Tokyo (JP); Tomohisa Kuzuyama, Tokyo (JP); Nobuo Mitsui, Nagano (JP)

Correspondence Address: GREENBLUM & BERNSTEIN, P.L.C., 1950 ROLAND CLARKE PLACE, RESTON, VA, 20191, US

(21) Appl. No.: 10/204,434

(22) PCT Filed: Feb. 28, 2001
(86) PCT No.: PCT/JP01/01501

(30) Foreign Application Priority Data
Mar. 2, 2000 (JP) 2000-56753

(51) Int. Cl. C12Q 1/68; C12Q 1/48; C12Q 1/42; C12Q 1/18; C12N 1/21

(52) U.S. Cl. 435/6; 435/32; 435/252.31; 435/21

(57) ABSTRACT

The present invention provides a method for screening substances which specifically inhibit the non-mevalonate pathway by using an organism capable of using both the mevalonate pathway and the non-mevalonate pathway as biosynthetic pathways of isopentenyl diphosphate (IPP) and dimethylallyl diphosphate (DMAPP). According to the present invention, it is possible to provide a novel method for screening anti-bacterial agents, herbicides or anti-malarial agents which can specifically inhibit any of the enzyme reactions on the non-mevalonate pathway for the synthesis of isopentenyl diphosphate and dimethylallyl diphosphate (DMAPP).

### TECHNICAL FIELD

The present invention relates to a method for screening substances which specifically inhibit the non-mevalonate pathway. More specifically, the present invention relates to a method for screening substances which specifically inhibit the non-mevalonate pathway by using an organism capable of using both the mevalonate pathway and the non-mevalonate pathway, a transformant used for the above screening method, and the substances useful as anti-bacterial agent, herbicide or anti-malarial agent selected by the above screening method.

### BACKGROUND ART

In many eubacteria including Bacillus subtilis or the plastid of higher plants, only the non-mevalonate pathway functions as a biosynthetic pathway of isopentenyl diphosphate (IPP) and dimethylallyl diphosphate (DMAPP) which are essential compounds for growth (Comprehensive Natural Products Chemistry. Elsevier, Amsterdam, Vol. 2, 45, 1999). When the non-mevalonate pathway of the above organisms is blocked, these organisms cannot carry out the biosynthesis of isopentenyl diphosphate (IPP) or dimethylallyl diphosphate (DMAPP), and accordingly the organisms become unable to grow. Therefore, a compound having an activity of inhibiting any enzyme on the non-mevalonate pathway is useful as anti-bacterial agent, herbicide or anti-malarial agent.

Regarding the non-mevalonate pathway, the first stage wherein 1-deoxy-D-xylulose-5-phosphate (DXP) is generated by condensation of pyruvic acid and glyceraldehyde-3-phosphate (Proc. Natl. Acad. Sci. USA, 94, 12857, (1997)), the second stage wherein 2-C-methyl-D-erythritol 4-phosphate (MEP) is generated from DXP (Tetrahedron Lett., 39, 4509, (1998)), the third stage wherein 4-(cytidine 5′-diphospho)-2-C-methyl-D-erythritol (CDP-ME) is generated from MEP (Tetrahedron Lett., 41, 703, (2000)), and the fourth stage wherein 2-phospho-4-(cytidine 5′-diphospho)-2-C-methyl-D-erythritol (CDP-ME2P) is generated from CDP-ME (Tetrahedron Lett., under contribution) have been clarified, and enzymes catalyzing each of the above reactions and genes encoding the enzymes have been identified. However, there are no findings regarding a reaction pathway from the fourth stage to IPP, enzymes catalyzing such reactions, and genes encoding those enzymes.

Presently, there is a report regarding a method for screening a compound which has an activity of specifically inhibiting DXP reductoisomerase that is an enzyme at the second stage on the non-mevalonate pathway (Proc. Natl. Acad. Sci. USA 95, 9879, (1998)). However, there are no reports regarding a method for screening compounds having an activity which inhibits an enzyme at other stages on the non-mevalonate pathway. A method of purifying each enzyme on the non-mevalonate pathway and screening compounds having an activity of inhibiting each enzyme may be considered, but the operation of purifying each enzyme and analyzing an activity of inhibiting the enzyme is complicated, and such an operation cannot be an efficient screening method. Moreover, since an enzyme catalyzing enzyme reactions after 2-phospho-4-(cytidine 5′-diphospho)-2-C-methyl-D-erythritol (CDP-ME2P) has not been identified at the moment, a substance which inhibits these enzyme reactions cannot be screened by the above described method.

As another biosynthetic pathway of IPP which is different from the non-mevalonate pathway, there is known the mevalonate pathway. Specifically, in eukaryote such as animals or yeast, IPP is biosynthesized from acetyl CoA via mevalonic acid. As stated above, many eubacteria including Bacillus subtilis or the plastid of higher plants do not use the mevalonate pathway, but use the non-mevalonate pathway.

It is known that Streptomyces, Streptomyces sp. CL190 (J. Antibiot. 43:444, 1990) synthesizes IPP via mevalonic acid (Tetrahedron Lett. 31:6025, 1990 and Tetrahedron Lett. 37:7979, 1996). Up to now, the present inventors have cloned a gene (hmgr) which encodes 3-hydroxy-3-methylglutaryl-CoA(HMG-CoA) reductase, which is an enzyme catalyzing one reaction on the mevalonate pathway, from Streptomyces sp. CL190 (J. Bacteriol. 181:1256, 1999).

### DISCLOSURE OF THE INVENTION

The first object to be achieved by the present invention is to provide a novel method for screening anti-bacterial agent, herbicide or anti-malarial agents which can specifically inhibit any enzyme reaction on the non-mevalonate pathway for synthesizing isopentenyl diphosphate and dimethylallyl diphosphate.

The second object to be achieved by the present invention is to provide an efficient method for screening anti-bacterial agent, herbicide or anti-malarial agent which can specifically inhibit any enzyme reaction on the non-mevalonate pathway for synthesizing isopentenyl diphosphate and dimethylallyl diphosphate.

The third object to be achieved by the present invention is to provide a method for screening anti-bacterial agent, herbicide or anti-malarial agents which can specifically inhibit an unknown enzyme reaction on the non-mevalonate pathway for synthesizing isopentenyl diphosphate and dimethylallyl diphosphate.

The fourth object to be achieved by the present invention is to provide a method for screening anti-bacterial agent, herbicide or anti-malarial agents which can specifically inhibit an enzyme reaction on the non-mevalonate pathway for synthesizing isopentenyl diphosphate and dimethylallyl diphosphate, by using a group of Streptomyces-derived genes which encode the mevalonate pathway.

As a result of intensive research to achieve the aforementioned objects, the present inventors have first integrated, into Bacillus subtilis, DNA encoding mevalonate kinase, phosphomevalonate kinase, diphosphomevalonate decarboxylase and IPP isomerase, i.e., an enzyme which catalyzes the conversion of isopentenyl diphosphate (IPP) and dimethylallyl diphosphate (DMAPP), which are a group of Streptomyces-derived genes involved in the mevalonate pathway. Then, these genes were induced with IPTG to produce a transformant of Bacillus subtilis which can use the mevalonate pathway in the presence of mevalonic acid. Subsequently, the growth of Bacillus subtilis has been analyzed by culturing the thus obtained transformant of Bacillus subtilis together with fosmidomycin (a test substance) in the presence of mevalonic acid and IPTG (i.e. under an environment where the mevalonate pathway can be used) or in the absence of mevalonic acid and IPTG (i.e. under an environment where the mevalonate pathway cannot be used). As a result, it was confirmed that fosmidomycin does not show an anti-bacterial activity in the presence of mevalonic acid and IPTG, but shows an anti-bacterial activity in the absence of mevalonic acid and IPTG. The present invention has been completed on the basis of these findings.

Thus, according to the present invention, there is provided a method for screening substances which specifically inhibit the non-mevalonate pathway by using an organism capable of using both the mevalonate pathway and the non-mevalonate pathway as biosynthetic pathways of isopentenyl diphosphate (IPP) and dimethylallyl diphosphate (DMAPP).

In one embodiment of the present invention, the organism capable of using both the mevalonate pathway and the non-mevalonate pathway as biosynthetic pathways of isopentenyl diphosphate (IPP) and dimethylallyl diphosphate (DMAPP) is an organism capable of using the mevalonate pathway only in the presence of mevalonic acid.

Preferably, the organism capable of using the mevalonate pathway only in the presence of mevalonic acid is a transformant obtained by introducing DNA encoding all enzymes necessary for using the mevalonate pathway in the presence of mevalonic acid, into a host having only the non-mevalonate pathway.

Preferably in the method of the present invention, the organism capable of using both the mevalonate pathway and the non-mevalonate pathway only in the presence of mevalonic acid is cultured with a test substance in the presence or absence of mevalonic acid, and test substances which do not inhibit the growth of the organism in the presence of mevalonic acid but inhibits the growth of the organism in the absence of mevalonic acid are selected as candidate substances.

Preferably, the enzymes necessary for using the mevalonate pathway in the presence of mevalonic acid are at least phosphomevalonate kinase, diphosphomevalonate decarboxylase, mevalonate kinase and IPP isomerase.

Preferably, the DNA encoding the enzymes necessary for using the mevalonate pathway in the presence of mevalonic acid is any of the followings:

(1) DNA having all nucleotide sequences of SEQ ID NOS: 9 to 12;

(2) DNA which has a deletion, substitution, addition and/or insertion of one or several nucleotides in the DNA of (1) above, and can allow the mevalonate pathway to function in the presence of mevalonic acid; or

(3) DNA which can hybridize with the DNA of (1) above under stringent condition, and can allow the mevalonate pathway to function in the presence of mevalonic acid.

In another embodiment of the present invention, the organism capable of using both the mevalonate pathway and the non-mevalonate pathway is an organism capable of using the mevalonate pathway in the absence of mevalonic acid.

Preferably, the organism capable of using the mevalonate pathway in the absence of mevalonic acid is a transformant obtained by introducing DNA encoding all enzymes necessary for using the mevalonate pathway in the absence of mevalonic acid, into a host having the non-mevalonate pathway.

Preferably, the organism capable of using both the mevalonate pathway and the non-mevalonate pathway and an organism incapable of using the mevalonate pathway but capable of using the non-mevalonate pathway are each cultured with a test substance, and test substances which do not inhibit the growth of the organism capable of using both the mevalonate pathway and the non-mevalonate pathway but inhibits the growth of the organism incapable of using the mevalonate pathway but capable of using the non-mevalonate pathway are selected as candidate substances.

Preferably, the enzymes necessary for using the mevalonate pathway in the absence of mevalonic acid are at least HMG-CoA synthase, HMG-CoA reductase, phosphomevalonate kinase, diphosphomevalonate decarboxylase, mevalonate kinase and IPP isomerase.

Preferably, the DNA encoding enzymes necessary for using the mevalonate pathway in the absence of mevalonic acid is any of the followings:

(1) DNA having a nucleotide sequence of SEQ ID NO: 13;

(2) DNA which has a deletion, substitution, addition and/or insertion of one or several nucleotides in the nucleotide sequence of SEQ ID NO: 13, and can allow the mevalonate pathway to function in the absence of mevalonic acid; or

(3) DNA which can hybridize with the DNA of (1) above under stringent condition, and can allow the mevalonate pathway to function in the absence of mevalonic acid.

In one embodiment of the present invention, an organism not having IPP isomerase is used as the organism.

The organism is preferably a microorganism, more preferably a bacterium, further preferably a Gram-positive bacterium, and particularly preferably Bacillus subtilis.

The method for screening according to the present invention is preferably used for the screening of candidate compounds for anti-bacterial agent, herbicide or anti-malarial agents.

According to another aspect of the present invention, there is provided a transformant which is obtained by introducing DNA encoding all enzymes necessary for using the mevalonate pathway as a biosynthetic pathway of isopentenyl diphosphate (IPP) and dimethylallyl diphosphate (DMAPP) in the presence of mevalonic acid, into a host having the non-mevalonate pathway.

Preferably, the enzymes necessary for using the mevalonate pathway as a biosynthetic pathway of isopentenyl diphosphate (IPP) and dimethylallyl diphosphate (DMAPP) in the presence of mevalonic acid are at least phosphomevalonate kinase, diphosphomevalonate decarboxylase, mevalonate kinase and IPP isomerase.

Preferably, the DNA encoding the enzymes necessary for using the mevalonate pathway in the presence of mevalonic acid is any of the followings:

(1) DNA having all nucleotide sequences of SEQ ID NOS: 9 to 12;

(2) DNA which has a deletion, substitution, addition and/or insertion of one or several nucleotides in the DNA of (1) above, and can allow the mevalonate pathway to function in the presence of mevalonic acid; or

(3) DNA which can hybridize with the DNA of (1) above under stringent condition, and can allow the mevalonate pathway to function in the presence of mevalonic acid.

According to further another aspect of the present invention, there is provided substances which specifically inhibit the non-mevalonate pathway, which are selected by the aforementioned screening method of the present invention. The aforementioned substances which specifically inhibit the non-mevalonate pathway are preferably substances having anti-bacterial activity, herbicide activity or anti-malarial activity.

Best Mode for Carrying Out the Invention

The embodiments and methods for carrying out the present invention will be explained in detail below.

(A) The Embodiment of the Screening Method of the Present Invention

The method for screening substances which specifically inhibit the non-mevalonate pathway according to the present invention is characterized in that it uses an organism capable of using both the mevalonate pathway and the non-mevalonate pathway as biosynthetic pathways of isopentenyl diphosphate (IPP) and dimethylallyl diphosphate (DMAPP).

The kind of the “organism” in the present specification is not particularly limited, but it is preferably an organism with which it can be simply examined whether or not a test substance has an activity of specifically inhibiting the non-mevalonate pathway. Specifically, a microorganism or cultured cell is preferable. The kind of an microorganism is not particularly limited, but it is preferably a bacterium, more preferably a Gram-positive bacterium, and particularly preferably Bacillus subtilis (Basillus). Moreover, a cultured cell derived from a plant is preferable as a cultured cell.

In the screening method of the present invention, there is used an organism capable of using both the mevalonate pathway and the non-mevalonate pathway. A transformant capable of using both the mevalonate pathway and the non-mevalonate pathway can be constructed by introducing DNA encoding enzymes which allow the mevalonate pathway to function into a host with using a bacterium originally having the non-mevalonate pathway as the host.

As an organism capable of using both the mevalonate pathway and the non-mevalonate pathway as biosynthetic pathways of isopentenyl diphosphate (IPP) and dimethylallyl diphosphate (DMAPP), there may used an organism capable of using the mevalonate pathway only in the presence of mevalonic acid d, or there may be used an organism capable of using the mevalonate pathway even in the absence of mevalonic acid.

Examples of the organism capable of using the mevalonate pathway only in the presence of mevalonic acid include a transformant which is obtained by introducing DNA encoding all enzymes necessary for using the mevalonate pathway in the presence of mevalonic acid into a host having the non-mevalonate pathway. The term “enzymes necessary for using the mevalonate pathway in the presence of mevalonic acid” used herein comprises at least phosphomevalonate kinase, diphosphomevalonate decarboxylase, mevalonate kinase and IPP isomerase. Examples of DNA encoding these enzymes include:

(1) DNA having all nucleotide sequences of SEQ ID NOS: 9 to 12;

(2) DNA which has a deletion, substitution, addition and/or insertion of one or several nucleotides in the DNA of (1) above, and can allow the mevalonate pathway to function in the presence of mevalonic acid; or

(3) DNA which can hybridize with the DNA of (1) above under stringent condition, and can allow the mevalonate pathway to function in the presence of mevalonic acid.

Moreover, examples of the organism capable of using the mevalonate pathway in the absence of mevalonic acid include a transformant which is obtained by introducing DNA encoding all enzymes necessary for using the mevalonate pathway in the absence of mevalonic acid into a host having the non-mevalonate pathway. The term “enzymes necessary for using the mevalonate pathway in the absence of mevalonic acid” used herein comprises at least HMG-CoA synthase, HMG-CoA reductase, phosphomevalonate kinase, diphosphomevalonate decarboxylase, mevalonate kinase and IPP isomerase. Examples of DNA encoding these enzymes include:

(1) DNA having a nucleotide sequence of SEQ ID NO: 13;

(2) DNA which has a deletion, substitution, addition and/or insertion of one or several nucleotides in the nucleotide sequence of SEQ ID NO: 13, and can allow the mevalonate pathway to function in the absence of mevalonic acid; or

(3) DNA which can hybridize with the DNA of (1) above under stringent condition, and can allow the mevalonate pathway to function in the absence of mevalonic acid.

The above-described DNA sequence includes genes which encode enzymes involved in the mevalonate pathway existing in Streptomyces, and the cloning of the genes is described in Japanese Patent Application No. 11-348375 (filed Dec. 8, 1999) (All contents described in Japanese Patent Application No. 11-348375 are incorporated herein by reference in their entirety.)

The term “a deletion, substitution, addition and/or insertion of one or several nucleotides” in the present specification means that any given number of nucleotides, for example, 1 to 20, preferably 1 to 15, more preferably 1 to 10, and further preferably 1 to 5 nucleotides are deleted, substituted, added and/or inserted.

The term “DNA which can hybridize with . . . under stringent condition” in the present specification means DNA which is obtained by using colony hybridization method, plaque hybridization method or Southern blot hybridization method using the DNA as a probe. Specifically, such DNA can be identified by performing hybridization at 65° C. in the presence of 0.7 to 1.0 M NaCl using a filter on which DNA derived from a colony or plaque or its DNA fragment is fixed, and then washing the filter with about 0.1 to 2×SSC solution (the composition of 1×SSC solution is 150 mM sodium chloride and 15 mM sodium citrate) under a condition of 65° C. The hybridization can be carried out according to the method described in Molecular Cloning: A Laboratory Manual, 2^{nd} Ed., Cold Spring Harbor Laboratory, Cold Spring Harbor, N.Y., 1989 (hereinafter, abbreviated as “Molecular Cloning 2^{nd} Ed”), etc.

Examples of DNA which can hybridize under stringent condition include DNA having a certain or more homology with the nucleotide sequence of DNA used as a probe, and the homology is for example 60% or more, preferably 70% or more, more preferably 80% or more, further preferably 90% or more, particularly preferably 95% or more, and most preferably 98% or more.

The “mevalonate pathway” in the present specification is a pathway in which IPP is biosynthesized by the steps of:

(1) converting acetoacetyl CoA into HMG-CoA (catalyzed by HMG-CoA synthase),

(2) converting HMG-CoA into mevalonic acid (catalyzed by HMG-CoA reductase),

(3) converting mevalonic acid into pyrophosphomevalonic acid (catalyzed and controlled by mevalonate (MVA) kinase and phosphomevalonate (PMVA) kinase), and

(4) converting pyrophosphomevalonic acid into isopentenyl pyrophosphate (IPP) (catalyzed by diphosphomevalonate (PMVA) decarboxylase), and a pathway in which DMAPP is further biosynthesized by the step of:

(5) converting IPP into DMAPP (catalyzed by IPP isomerase).

Accordingly, examples of enzymes necessary for allowing the mevalonate pathway to function in the presence of mevalonic acid include at least PMVA kinase, PMVA decarboxylase, MVA kinase and IPP isomerase, and enzymes necessary for allowing the mevalonate pathway to function in the absence of mevalonic acid include HMG-CoA reductase and HMG-CoA synthase in addition to the above mentioned enzymes.

(B) Method for Constructing an Organism Capable of Using Both the Mevalonate Pathway and the Non-Mevalonate Pathway as Biosynthetic Pathways of Isopentenyl Diphosphate (IPP) and Dimethylallyl Diphosphate (DMAPP)

Up till now, the present inventors have cloned a gene (hmgr) which encodes an enzyme which catalyzes one reaction on the mevalonate pathway, 3-hydroxy-3-methylglutaryl-CoA (HMG-CoA) reductase, from an actinomycete, Streptomyces sp. CL190 (J. Bacteriol. 181:1256, 1999). The DNA having a nucleotide sequence of SEQ ID NO: 13 in the present specification can be obtained by using this hmgr gene as a probe. Specifically, the DNA can be obtained by the following procedure.

Streptomyces sp. CL 190 is cultured in an appropriate medium such as a GPY medium (1% glucose, 0.4% polypeptone, 0.4% yeast extract, 0.5% MgSO₄·7H₂O, 0.1% K₂HPO₄) at an appropriate temperature (e.g. 30° C.) for several days. After culturing, cells are collected from the obtained culture solution by centrifugal separation, and chromosomal DNA is then isolated and purified from the cells according to a conventional method (Molecular Cloning 2^{nd} Ed). The thus obtained chromosomal DNA is cleaved with an appropriate restriction enzyme (e.g. SnaBI), and is then subjected to Southern hybridization using the hmgr gene as a probe (Molecular Cloning 2^{nd} Ed). As a result of Southern hybridization, the signal of a probe is detected at a specific position (for example, when SnaBI is used as a. restriction enzyme for the digestion of chromosomal DNA, the signal is detected at the position of 6.7 kb).

Subsequently, the chromosomal DNA of Streptomyces sp. CL 190 is cleaved again with the same restriction enzyme (e.g. SnaBI) as described above, and is then subjected to agarose gel electrophoresis. As a result of Southern hybridization, the signal of a probe is detected at a specific position (when SnaBI is used as a restriction enzyme, the signal is detected at the position of 6.7 kb). A DNA fragment corresponding to the position is extracted and collected from the agarose gel. The collected DNA fragment is blunt-ended by using T4 DNA polymerase (purchased from Takara Shuzo Co., Ltd.), and then inserted into an appropriate plasmid (e.g. pUC118, etc.) to produce the chromosomal DNA library of Streptomyces sp. CL 190. Using this chromosomal DNA library, a suitable host (e.g. E. coli JM 109, etc.) is transformed according to a conventional method (Molecular Cloning 2^{nd} Ed), and the transformants are screened by a colony hybridization method using the hmgr gene as a probe, so that the transformant of E. coli having a plasmid containing the hmgr gene can be isolated. A plasmid is extracted from the isolated transformant according to a conventional method, so that a DNA fragment containing the hmgr gene can be isolated. An example of DNA isolated by the above-described method includes DNA having a nucleotide sequence of SEQ ID NO: 13.

Moreover, using the chromosomal DNA of Streptomyces sp. CL 190 as a template, PCR is carried out using sense primers and antisense primers having the combinations of the nucleotide sequences of SEQ ID NOS: 1 and 2, SEQ ID NOS: 3 and 4, SEQ ID NOS: 5 and 6, and SEQ ID NOS: 7 and 8, respectively, so as to amplify DNA fragments encoding phosphomevalonate kinase (SEQ ID NO: 9), diphosphomevalonate decarboxylase (SEQ ID NO: 10), mevalonate kinase (SEQ ID NO: 11) and Protein X (IPP isomerase) (SEQ ID NO: 12).

PCR can be carried out, for example, using TaKaRa LA-PCR™ Kit Ver. 2 (Takara Shuzo Co., Ltd.), Expand™ High-Fidelity PCR System (Boehringer Mannheim), or the like, and using DNA Thermal Cycler (Perkin Elmer Japan), etc. To facilitate the subsequent cloning operation, it is preferable to add appropriate restriction sites to primers.

SEQ ID NOS: 1, 3, 5 and 7 have a nucleotide sequence GGAGGTTGTTTT, which contains the SD sequence GGAGG of a penP gene (FEBS Letters 422, 243, (1998)) behind the restriction site. By adding this SD sequence, it becomes possible to allow the gene of Streptomyces to be expressed in Bacillus subtilis.

Examples of the reaction conditions for PCR include a condition that a reaction process of 94° C. to 95° C., 30 seconds (denaturation), 55° C. to 60° C., 30 seconds to 1 minute (annealing) and around 72° C., 1 to 2 minutes (extension) is defined as 1 cycle, and this process is repeated, for example, for 20 to 40 cycles, and then a reaction is carried out at 72° C. for 7 to 10 minutes. A DNA fragment can be cloned in an appropriate vector which can be amplified in E. coli. However, conditions for PCR are not limited to the above-described conditions, and changes and modifications can be made by a person skilled in the art as appropriate.

The cloning of the amplified DNA can be carried out by a conventional method such as one described in Molecular Cloning 2^{nd} Ed., Current Protocols in Molecular Biology, Supplement 1-38, John Wiley & Sons (1987-1997) (hereinafter, abbreviated as Current Protocols in Molecular Biology), DNA Cloning 1: Core Techniques, A Practical Approach, Second Edition, Oxford University Press (1995), etc. or by using a commercially available kit such as SuperScript Plasmid System for cDNA Synthesis and Plasmid Cloning (Life Technology) or ZAP-cDNA Synthesis Kit (Staratagene).

Any vector such as phage vector or plasmid vector can be used as a cloning vector, as long as it can autonomously replicate in E. coli K12. An E. coli expression vector may be used as a cloning vector. Specific examples include ZAP Express [Stratagene, Strategies, 5, 58 (1992)], pBluescript II SK(+) [Nucleic Acids Research, 17, 9494(1989)], Lambda ZAP II (Stratagene), λ gt10, λ gt11 [DNA Cloning, A Practical Approach, 1, 49(1985)], λ Trip1Ex (Clonetech), λ ExCell (Pharmacia), pT7T318U (Pharmacia), pcD2 [Mol. Cen. Biol., 3, 280 (1983)], pMW218 (Wako Pure Chemical Industries, Ltd.), pUC118 (Takara Shuzo Co., Ltd.), pEG400 [J. Bac., 172, 2392 (1990)], and pQE-30 (QIAGEN).

A plasmid containing DNA of interest can be obtained from the obtained transformant according to a conventional method such as one described in Molecular Cloning 2^{nd} Ed., Current Protocols in Molecular Biology, DNA Cloning 1: Core Techniques, A Practical Approach, Second Edition, Oxford University Press (1995), etc. By the above-described method, DNA having a nucleotide sequence of SEQ ID NOS: 9, 10, 11 and 12 can be obtained.

Moreover, in the present invention, there can also be used DNA which has a deletion, substitution, addition and/or insertion of one or several nucleotides in the DNA having the nucleotide sequence of SEQ ID NOS: 9 to 12, and can allow the mevalonate pathway to function in the presence of mevalonic acid; or DNA which can hybridize with the DNA having the nucleotide sequence of SEQ ID NOS: 9 to 12 under stringent condition, and can allow the mevalonate pathway to function in the presence of mevalonic acid.

Such DNA can be isolated by using the nucleotide sequence of a DNA fragment derived from Streptomyces having the nucleotide sequence of SEQ ID NOS: 9 to 12 and screening for the homolog of the DNA from other microorganisms or the like under appropriate condition, or it can be produced by a method known to a person skilled in the art such as chemical synthesis, genetic engineering or mutagenesis.

For example, such DNA can be obtained by a method of contacting DNA having the nucleotide sequence of SEQ ID NOS: 9 to 12 with an agent of mutagene, a method of applying ultraviolet ray to the DNA, a method of genetic engineering technique, or the like. The site-directed mutagenesis which is one of the genetic engineering technique is useful as a technique for introducing a specific mutation into a specific position, and can be carried out according to a method described in Molecular Cloning 2^{nd} Ed.; Current Protocols in Molecular Biology; Nucleic Acids Research, 10, 6487, 1982; Nucleic Acids Research, 12, 9441, 1984; Nucleic Acids Research, 13, 4431, 1985; Nucleic Acids Research, 13, 8749, 1985; Proc. Natl. Acad. Sci. USA, 79, 6409, 1982; Proc. Natl. Acad. Sci. USA, 82, 488, 1985; Gene, 34, 315, 1985; Gene, 102, 67, 1991; etc.

To allow the thus obtained DNA to be expressed in a host cell, the DNA fragment of interest is first cleaved with a restriction enzyme or deoxyribonuclease into the appropriate length of a DNA fragment comprising the gene. The obtained DNA fragment is inserted downstream of a promoter in an expression vector, and then the expression vector into which the above DNA is inserted is introduced into a host cell suitable for the expression vector.

The DNA fragments encoding phosphomevalonate kinase (SEQ ID NO: 9), diphosphomevalonate decarboxylase (SEQ ID NO: 10), mevalonate kinase (SEQ ID NO: 11) and IPP isomerase (SEQ ID NO: 12) may be incorporated into expression vectors separately, and then the obtained 4 types of expression vectors may be introduced into a host. Otherwise, 2 types or more, preferably 3 types or more, and more preferably all the 4 types of the above DNA fragments may be cloned into a single expression vector and then this expression vector may be introduced into a host. From the viewpoint of operability, it is preferable that an expression vector into which the above 4 types of DNA fragments are incorporated is introduced into a host.

The type of a host is not particularly limited, as long as it has the non-mevalonate pathway. Preferred type of a host is a bacterium or a plant cell. Examples of a bacterium include a Gram-positive bacterium (Micrococcus sp., Bacillus sp., Clostridium sp., Corynebacterium sp., etc.), and a Gram-negative bacterium (Rhodopseudomonas sp., Pseudomonas sp., Escherichia sp., Salmonella sp., Thiobacillus sp., Neisseria sp., etc.). Since a Gram-positive bacterium has a higher efficiency of taking a substance into a cell than a Gram-negative bacterium, the Gram-positive bacterium is preferable.

As an expression vector, there is used a vector which can autonomously replicate in the above host cell or can be incorporated into a chromosome and which comprises a promoter in a position where the above DNA of interest can be transcribed.

Where a bacterium is used as a host cell, the expression vector for expressing the above DNA is capable of autonomous replication in the bacterium, and also is preferred to be a recombinant vector composed of a promoter, a ribosome binding sequence, the above DNA and a transcription termination sequence. A gene which regulates the promoter may also be contained therein.

Examples of an expression vector include pAA101 (Amersham), pBTrP2, pBTac1, pBTac2 (all of these are commercially available from Boehringer Mannheim), pKK233-2 (Pharmacia), pSE280 (Invitrogen), pGEMEX-1 (Promega), pQE-8 (QIAGEN), pQE-30 (QIAGEN), pKYP10 (Japanese Patent Application Laying-Open (Kokai) No. 58-110600), pKYP200 [Agrc. Biol. Chem., 48, 669 (1984)], PLSA1 [Agrc. Biol. Chem., 53, 277 (1989)], pGEL1 [Proc. Natl. Acad. Sci. USA, 82, 4306 (1985)], pBluescriptII SK+, pBluescriptII SK(−) (Stratagene), pTrS30 (FERMBP-5407), pTrS32 (FERM BP-5408), pGEX (Pharmacia), pET-3 (Novagen), pTerm2 (U.S. Pat. No. 4,686,191, U.S. Pat. No. 4,939,094, U.S. Pat. No. 5,160,735), pSupex, pUB110, pTP5, pC194, pUC18 [Gene, 33, 103 (1985)], pUC19 [Gene, 33, 103 (1985)], pSTV28 (Takara Shuzo Co., Ltd.), pSTV29 (Takara Shuzo Co., Ltd.), pUC118 (Takara Shuzo Co., Ltd.), pPA1 (Japanese Patent Application Laying-Open (Kokai) No. 63-233798), pEG400 [J. Bacteriol., 172, 2392 (1990)], and pQE-30 (QIAGEN).

Any promoter can be used, as long as expression can be carried out in a host cell. Examples of a promoter include a promoter derived from Escherichia coli or phage, such as a trp promoter (P trp), lac promoter (P lac), PL promoter, PR promoter or P_{SE} promoter, SP01 promoter, SP02 promoter, and penP promoter. Moreover, artificially designed or modified promoters such as a promoter obtained by connecting two P trp promoters in series (P trp×2), tac promoter, letl promoter and lacT7 promoter, can also be used.

As a method of introducing a recombinant vector, any method can be applied as long as it is a method of introducing DNA into the above host cell. Examples of such a method include a method using calcium ion [Proc. Natl. Acad. Sci. USA, 69, 2110 (1972)], the protoplast method (Japanese Patent Application Laying-Open (Kokai) No. 63-2483942), and a method described in Gene, 17, 107 (1982) or Molecular & General Genetics, 168, 111 (1979).

The thus produced transformant of the present invention can be cultured in a medium according to a common method used for the culture of a host.

The medium may be either a natural medium or a synthetic medium, as long as the medium contains a carbon source, a nitrogen source, inorganic salts or the like that can be assimilated by the transformant of the present invention, and enables efficient culturing of the transformant.

Any carbon source may be used, as long as it can be assimilated by each microorganism. Examples thereof include carbohydrate such as glucose, fructose, sucrose, molasses containing them, starch or starch hydrolysate; organic acid such as acetic acid or propionic acid; and alcohols such as ethanol and propanol. Examples of a nitrogen source include ammonia; ammonium salts of various inorganic or organic acids such as ammonium chloride, ammonium sulfate, ammonium acetate and ammonium phosphate; other nitrogen-containing compounds; and peptone, meat extract, yeast extract, corn steep liquor, casein hydrolysate, soybean cake and soybean cake hydrolysate, various fermentative cell bodies and digests thereof; etc. Examples of an inorganic product include potassium monophosphate, potassium diphosphate, magnesium phosphate, magnesium sulfate, sodium chloride, ferrous sulfate, manganese sulfate, copper sulfate, and calcium carbonate.

Culturing can be carried out under aerobic conditions such as shaking culturing or deep aeration and agitation culturing. The culturing temperature is preferably 15° C. to 40° C., and the culturing period is generally 16 hours to 7 days. The pH in culturing is retained at 3.0 to 9.0. The control of pH is carried out using inorganic or organic acid, an alkali solution, urea, calcium carbonate, ammonia, etc.

In addition, antibiotics such as ampicillin or tetracycline may be added to a medium during culturing as necessary.

When a microorganism transformed with an expression vector comprising an inductive promoter as a promoter is cultured, an inducer may be added to a medium as necessary. For example, when a microorganism transformed with an expression vector comprising a lac promoter is cultured, isopropyl- β-D-thiogalactopyranoside (IPTG) or the like may be added to a medium, and when a microorganism transformed with an expression vector comprising a trp promoter is cultured, indoleacrylic acid (IAA) or the like may be added thereto.

(C) Method for Screening Substances Specifically Inhibiting the Non-Mevalonate Pathway by Using an Organism Capable of Using Both the Mevalonate Pathway and the Non-Mevalonate Pathway as Biosynthetic Pathways of Isopentenyl Diphosphate (IPP) and Dimethylallyl Diphosphate (DMAPP)

An organism capable of using both the mevalonate pathway and the non-mevalonate pathway which is produced in (B) above is cultured with a test substance under both an environment where the mevalonate pathway can be used and an environment where the mevalonate pathway cannot be used, and then a test substance which inhibits the growth of the organism under an environment where the mevalonate pathway cannot be used, but does not inhibit the growth of the organism under an environment where the mevalonate pathway can be used, is selected. By this method, a substance having an activity of specifically inhibiting the non-mevalonate pathway can be identified.

Embodiments of culturing an organism with a test substance under an environment where the mevalonate pathway can be used and under an environment where the pathway cannot be used, include, for example, the following two types.

In the first embodiment, using an organism having enzymes which catalyze the reaction after mevalonic acid on the mevalonate pathway, the organism is cultured with a test substance both in a medium containing mevalonic acid (an environment where the mevalonate pathway can be used) and in a medium containing no mevalonic acid (an environment where the mevalonate pathway cannot be used).

In the second embodiment, two types of organisms, that is, an organism having all enzymes which catalyze all processes of converting acetoacetyl CoA into isopentenyl pyrophosphate (IPP) and dimethylallyl diphosphate (DMAPP) by way of HMG-CoA, mevalonic acid and pyrophosphomevalonic acid (i.e. an organism capable of using the mevalonate pathway in the absence of mevalonic acid), and an organism incapable of using the mevalonate pathway, are cultured with a test substance in the same medium.

In the present invention, the above-described first embodiment is particularly preferable.

Moreover, in the present invention, the above screening method can be carried out using an organism wherein IPP isomerase gene is destructed. By performing screening using such a mutant organism, it also becomes possible to screen inhibitors which are specific for the reaction of synthesizing isopentenyl diphosphate (IPP) or dimethylallyl diphosphate (DMAPP) from an intermediate substance on the non-mevalonate pathway. That is to say, a transformant is prepared by introducing DNA fragments encoding enzymes existing on the mevalonate pathway, i.e., phosphomevalonate kinase, diphosphomevalonate decarboxylase, mevalonate kinase and IPP isomerase, into an organism wherein IPP isomerase gene is destructed. This transformant is then allowed to grow in the presence of a test substance under the two conditions of: (1) mevalonic acid is absent and the introduced IPP isomerase gene is not expressed; and (2) mevalonic acid is present and all of the introduced genes of the enzymes on the mevalonate pathway are expressed. By selecting a test substance with which the transformant cannot grow under the conditions of (1) above but can grow under the conditions of (2) above, a substance which specifically inhibits any enzyme. involved in the non-mevalonate pathway leading to IPP or DMAPP can be screened.

Fosmidomycin is an inhibitor which is specific for DXP reductoisomerase that is an enzyme at the second stage on the non-mevalonate pathway (Tetrahedron Lett. 39, 7913, (1998); Science 285, 1573, (1999)), and is a compound having an anti-bacterial activity (J. Antimicrob. Chemother. 24, 131, (1989)), a herbicide activity (U.S. Pat. No. 4,693,742) or an anti-malarial activity (Science 285, 1573, (1999)). In the examples of the present specification set forth later, the growth of Bacillus subtilis has been analyzed by culturing Bacillus subtilis, which can use the mevalonate pathway in the presence of mevalonic acid and IPTG and also can use the non-mevalonate pathway, together with fosmidomycin (a test substance) in the presence of mevalonic acid and IPTG (i.e. under an environment where the mevalonate pathway can be used) or in the absence of mevalonic acid and IPTG (i.e. under an environment where the mevalonate pathway cannot be used). As a result, it was confirmed that fosmidomycin does not show an anti-bacterial activity in the presence of mevalonic acid and IPTG but shows an anti-bacterial activity in the absence of mevalonic acid and IPTG. From this result, it was demonstrated that the screening method of the present invention was useful for screening substances which specifically inhibit the non-mevalonate pathway.

The screening method of the present invention is suitable for examining various kinds of compounds for a substance which is likely to specifically inhibit the non-mevalonate pathway, and the type of the test substance is not particularly limited. Where the first screening is carried out on test substances using the screening method of the present invention, various compound libraries can be used. Examples of such compound libraries include a combinatorial library, a natural product library, other small molecule compound libraries, etc. Moreover, a commercially available compound from a commercial source can also be screened.

Examples of a test substance include a low molecular weight compound, and peptide.

The present invention will be explained further in detail below, but the present invention is not limited by the examples.

### EXAMPLES

### Example 1

### Obtainment of DNAs Encoding Proteins Involved in the Mevalonate Pathway Which Functions in Bacillus subtilis

According to a conventional method, chromosomal DNA was isolated and purified from Streptomyces sp. CL190. Sense primers and antisense primers having each of the combinations of the nucleotide sequences of SEQ ID NOS: 1 and 2, SEQ ID NOS: 3 and 4, SEQ ID NOS: 5 and 6, and SEQ ID NOS: 7 and 8, were synthesized using a DNA synthesizer.

Using the sense primers and the antisense primers having each of the combinations of the nucleotide sequences of SEQ ID NOS: 1 and 2, SEQ ID NOS: 3 and 4, SEQ ID NOS: 5 and 6, and SEQ ID NOS: 7 and 8 separately, the following PCR is carried out using the chromosomal DNA of the CL190 strain as a template, so as to amplify DNA fragments each encoding each of phosphomevalonate kinase (SEQ ID NO: 9), diphosphomevalonate decarboxylase (SEQ ID NO: 10), mevalonate kinase (SEQ ID NO: 11) and IPP isomerase (SEQ ID NO: 12).

Moreover, as a restriction site, SEQ ID NO: 1 has HindIII at the 5′-terminus, SEQ ID NOS: 2 and 3 have XbaI at the 5′-terminus, SEQ ID NOS: 4 and 5 have BgIII at the 5′-terminus, SEQ ID NOS: 6 and 7 have ClaI at the 5′-terminus, and SEQ ID NO: 8 has SphI at the 5′-terminus.

Furthermore, SEQ ID NOS: 1, 3, 5 and 7 have a nucleotide sequence, GGAGGTTGTTTT, which comprises the SD sequence GGAGG of penP gene (FEBS Letters 422, 243, (1998)) behind the restriction site. By adding this SD sequence, the gene of Streptomyces can be expressed in Bacillus subtilis.

Using chromosomal DNA as a template, PCR was carried out using these primers and Expand™ High-Fidelity PCR System (Boehringer Mannheim). PCR was carried out by conducting reaction at 95° C. for 1 minute, repeating the cycles of 95° C. for 30 seconds, 60° C. for 30 seconds and 72° C. for 1 minute 30 times, and then conducting reaction 72° C. for 10 minutes.

Each DNA fragment amplified by PCR was obtained by agarose gel electrophoresis, and after purification, the purified DNA fragment was dissolved in 5 μl of TE buffer.

To these solutions was added 0.5 μl of pGEM T-easy vector (Promega), and then ligation reaction was carried out to obtain recombinant DNAs.

Using this recombinant DNAs, Escherichia coli JM109 was transformed by a conventional method, and then the obtained transformants were applied on an LB agar medium containing 50 μg/ml ampicillin followed by culturing at 37° C. overnight.

Several colonies of the growing ampicillin-resistant transformants were subjected to shaking culturing at 37° C. overnight in 5 ml of LB liquid medium containing 50 μg/ml ampicillin.

The obtained culture solutions were centrifuged to obtain cells.

Plasmids were isolated from the obtained cells according to a conventional method.

The plasmids which were isolated by the above method were cleaved with various types of restriction enzymes to examine the structures and the nucleotide sequences were determined, and it was thus confirmed that these were plasmids into which the DNA fragments having correct nucleotide sequences of interest were inserted.

A plasmid containing DNA having the nucleotide sequence of SEQ ID NO: 9 was named as pGEMMVK1, a plasmid containing DNA having the nucleotide sequence of SEQ ID NO: 10 was named as pGEMMDC, a plasmid containing DNA having the nucleotide sequence of SEQ ID NO: 11 was named as pGEMMVK2, and a plasmid containing DNA having the nucleotide sequence of SEQ ID NO: 12 was named as pGEMHYP.

The pGEMMVK2 was treated with BgIII and ClaI, and a fragment having the nucleotide sequence of SEQ ID NO: 11 was obtained by agarose gel electrophoresis, and was then subcloned into an expression vector pAA101 (Amersham) by a conventional method. The plasmid obtained by this subcloning was named as pBMV1.

The pGEMHYP was treated with ClaI and SphI, and a fragment having the nucleotide sequence of SED ID NO: 12 was obtained by agarose gel electrophoresis, and was then subcloned into the pBMV1 by a conventional method. The plasmid obtained by this subcloning was named as pBMV2.

The pGEMMDC was treated with XbaI and BglII, and a fragment having the nucleotide sequence of SEQ ID NO: 10 was obtained by agarose gel electrophoresis, and was then subcloned into the pBMV2 according to a conventional method. The plasmid obtained by this subcloning was named as pBMV3.

The pGEMMVK1 was treated with HindIII and XbaI, and then subcloned into the pBMV3 according to a conventional method. The plasmid obtained by this subcloning was named as pBMV4.

### Example 2

### Screening of an Inhibitor on the Non-Mevalonate Pathway

According to a conventional method, the pBMV4 was introduced into B. subtilis 168 strain to obtain a transformant, B. subtilis 168 (pBMV4) which shows resistance to chloramphenicol with a concentration of 3.4 μg/ml. The B. subtilis 168 (pBMV4) was subjected to shaking culturing at 37° C. overnight in 5 ml of LB liquid medium containing 3.4 μg/ml chloramphenicol.

One thousandth amount of the above culture solution was added into Nutrient Broth “Eiken” (Eiken Chemical Co., Ltd.) containing 1 mM IPTG and 3.4 μg/ml chloramphenicol to prepare an agar medium.

One thousandth amount of the above culture solution was added into Nutrient Broth “Eiken” containing 1 mM IPTG, 3.4 μg/ml chloramphenicol and 0.02% mevalonic acid to prepare an agar medium. The gene on the pBMV4 is expressed only in the presence of IPTG 50 μl of aqueous solution of 5 μg/ml fosmidomycin was dropped on a paper disk, and then the paper disk was placed on the above two types of agar mediums, followed by culturing at 37° C. overnight.

After completion of the culturing, a growth inhibition ring due to fosmidomycin was not observed in a medium to which mevalonic acid and IPTG were added, whereas a growth inhibition ring due to fosmidomycin was observed in a medium to which mevalonic acid and IPTG were not added. The inhibition of the non-mevalonate pathway due to fosmidomycin could be confirmed by using media with or without mevalonic acid and IPTG, and B. subtilis 168 (pBMV4).

According to the similar method, by using a test compound instead of fosmidomycin, a substance which inhibits any enzyme reaction on the non-mevalonate pathway can be screened.

### Example 3

### Construction of IPP Isomerase Gene (ypgA) Disrupted Strain of Bacillus subtilis

The ypgA gene of Bacillus subtilis encodes IPP isomerase (Proc. Natl. Acad. Sici. USA., 98, 932-937, 2001).

Using the following primers which are specific for the ypgA gene: N-terminus primer (SEQ ID NO: 14) GGGGATCCTTGGAGCAAACGCACTGC C-terminus primer (SEQ ID NO: 15) GGGGATCCAAAAAAGGAGATTTGCCGC a partial region of the ypgA gene was amplified by PCR. Using these primers, the sequence corresponding to nucleotides 416 to 693 of the ypgA gene (1,057 nucleotides) can be amplified. After confirmation of the nucleotide sequence, this amplified DNA fragment was ligated to the BamHI site of a vector for Bacillus subtilis, pMUTIN2 (MICROBIOLOGY, 144: 3097-3104, 1998), and the constructed plasmid was named as pMYPGA.

The B. subtilis 168 was transformed with the pMYPGA, and an erythromycin-resistant clone was selected. In the erythromycin-resistant clone, it is predicted that the ypgA gene of the genome is destructed as a result of homologous recombination. It was confirmed by Southern hybridization that the ypgA gene of the obtained erythromycin-resistant clone was destructed. This IPP isomerase gene (ypgA) disrupted strain was named as B. subtilis delypgA.

### Example 4

### Screening of Inhibitors on the Non-Mevalonate Pathway

According to a conventional method, the pBMV4 was introduced into the B. subtilis delypgA strain produced in Example 3 so as to obtain a transformant B. subtilis delypgA (pBMV4) which shows resistance to chloramphenicol with a concentration of 3.4 μg/ml. The B. subtilis delypgA (pBMV4) was subjected to shaking culturing at 37° C. overnight in 5 ml of LB liquid medium containing 3.4 μg/ml chloramphenicol.

One thousandth amount of the above culture solution was added into Nutrient Broth “Eiken” (Eiken Chemical Co., Ltd.) containing 3.4 μg/ml chloramphenicol to prepare an agar medium.

One thousandth amount of the above culture solution was added into Nutrient Broth “Eiken” containing 1 mM IPTG, 3.4 μg/ml chloramphenicol and 0.02% mevalonic acid to prepare an agar medium. The gene on the pBMV4 is expressed only in the presence of IPTG

50 μl of aqueous solution of 5 μg/ml fosmidomycin was dropped on a paper disk, and then the paper disk was placed on the above two types of agar mediums, followed by culturing at 37° C. overnight.

After completion of the culture, a growth inhibition ring due to fosmidomycin was not observed in a medium to which mevalonic acid and IPTG were added, whereas the growth inhibition ring due to fosmidomycin was observed in a medium to which mevalonic acid and IPTG were not added. The inhibition of the non-mevalonate pathway due to fosmidomycin could be confirmed by using media with or without mevalonic acid and IPTG, and B. subtilis delypgA (pBMV4).

According to the similar method, by using a test compound instead of fosmidomycin, substances which inhibit any enzyme reaction on the non-mevalonate pathway can be screened.

Industrial Applicability

According to the present invention, it is possible to provide a novel method for screening anti-bacterial agents, herbicides or anti-malarial agents which can specifically inhibit any of enzyme reactions on the non-mevalonate pathway for synthesizing isopentenyl diphosphate. In the present invention, operations are simple and screening efficiency is high, since it is not necessary to purify an enzyme involved in the non-mevalonate pathway and determine its enzyme activity. Moreover, anti-bacterial agents, herbicides or anti-malarial agents which can specifically inhibit an unknown enzyme reaction on the non-mevalonate pathway can also be screened by the screening method of the present invention.

1. A method for screening substances which specifically inhibit the non-mevalonate pathway by using an organism capable of using both the mevalonate pathway and the non-mevalonate pathway as biosynthetic pathways of isopentenyl diphosphate (IPP) and dimethylallyl diphosphate (DMAPP).

2. The method of claims 1 wherein the organism capable of using both the mevalonate pathway and the non-mevalonate pathway as a biosynthetic pathway of isopentenyl diphosphate (IPP) and dimethylallyl diphosphate (DMAPP) is an organism capable of using the mevalonate pathway only in the presence of mevalonic acid.

3. The method of claims 2 wherein the organism capable of using the mevalonate pathway only in the presence of mevalonic acid is a transformant obtained by introducing DNA encoding all enzymes necessary for using the mevalonate pathway in the presence of mevalonic acid, into a host having the non-mevalonate pathway.

4. The method of any one of claims 1-3 wherein the organism capable of using both the mevalonate pathway and the non-mevalonate pathway only in the presence of mevalonic acid is cultured with a test substance in the presence or absence of mevalonic acid, and a test substance which does not inhibit the growth of the organism in the presence of mevalonic acid but inhibits the growth of the organism in the absence of mevalonic acid is selected as a candidate substance.

5. The method of claim 3 or 4 wherein the enzymes necessary for using the mevalonate pathway in the presence of mevalonic acid are at least phosphomevalonate kinase, diphosphomevalonate decarboxylase, mevalonate kinase and IPP isomerase.

6. The method of any one of claims 3-5 wherein the DNA encoding the enzymes necessary for using the mevalonate pathway in the presence of mevalonic acid is any of the followings:
(1) DNA having all nucleotide sequences of SEQ ID NOS: 9 to 12;
(2) DNA which has a deletion, substitution, addition and/or insertion of one or several nucleotides in the DNA of (1) above, and can allow the mevalonate pathway to function in the presence of mevalonic acid; or
(3) DNA which can hybridize with the DNA of (1) above under stringent condition, and can allow the mevalonate pathway to function in the presence of mevalonic acid.

7. The method of claim 1 wherein the organism capable of using both the mevalonate pathway and the non-mevalonate pathway is an organism capable of using the mevalonate pathway in the absence of mevalonic acid.

8. The method of claim 7 wherein the organism capable of using the mevalonate pathway in the absence of mevalonic acid is a transformant obtained by introducing DNA encoding all enzymes necessary for using the mevalonate pathway in the absence of mevalonic acid, into a host having the non-mevalonate pathway.

9. The method of claim 7 or 8 wherein the organism capable of using both the mevalonate pathway and the non-mevalonate pathway and an organism incapable of using the mevalonate pathway but capable of using the non-mevalonate pathway are each cultured with a test substance, and a test substance which does not inhibit the growth of the organism capable of using both the mevalonate pathway and the non-mevalonate pathway but inhibits the growth of the organism incapable of using the mevalonate pathway but capable of using the non-mevalonate pathway is selected as a candidate substance.

10. The method of claim 8 or 9 wherein the enzymes necessary for using the mevalonate pathway in the absence of mevalonic acid are at least HMG-CoA synthase, HMG-CoA reductase, phosphomevalonate kinase, diphosphomevalonate decarboxylase, mevalonate kinase and IPP isomerase.

11. The method of any one of claims 8 to 10 wherein the DNA encoding enzymes necessary for using the mevalonate pathway in the absence of mevalonic acid is any of the followings:
(1) DNA having a nucleotide sequence of SEQ ID NO: 13;
(2) DNA which has a deletion, substitution, addition and/or insertion of one or several nucleotides in the nucleotide sequence of SEQ ID NO: 13, and can allow the mevalonate pathway to function in the absence of mevalonic acid; or
(3) DNA which can hybridize with the DNA of (1) above under stringent condition, and can allow the mevalonate pathway to function in the absence of mevalonic acid.

12. The method of any one of claims 1-11 wherein an organism not having IPP isomerase is used as said organism.

13. The method of any one of claims 1 to 12 wherein the organism is a microorganism.

14. The method of claim 13 wherein the organism is a bacterium.

15. The method of claim 13 or 14 wherein the organism is a Gram-positive bacterium.

16. The method of any of claims 13 to 15 wherein the organism is Bacillus subtilis.

17. The method of any one of claims 1 to 16 which is used for the screening of candidate compounds for anti-bacterial agents, herbicides or anti-malarial agents.

18. A transformant which is obtained by introducing DNA encoding all enzymes necessary for using the mevalonate pathway as a biosynthetic pathway of isopentenyl diphosphate (IPP) and dimethylallyl diphosphate (DMAPP) in the presence of mevalonic acid, into a host having the non-mevalonate pathway.

19. The transformant of claim 18 wherein the enzymes necessary for using the mevalonate pathway as a biosynthetic pathway of isopentenyl diphosphate (IPP) and dimethylallyl diphosphate (DMAPP) in the presence of mevalonic acid are at least phosphomevalonate kinase, diphosphomevalonate decarboxylase, mevalonate kinase and IPP isomerase.

20. The transformant of claim 18 or 19 wherein the DNA encoding the enzymes necessary for using the mevalonate pathway in the presence of mevalonic acid is any of the followings:
(1) DNA having all nucleotide sequences of SEQ ID NOS: 9 to 12;
(2) DNA which has a deletion, substitution, addition and/or insertion of one or several nucleotides in the DNA of (1) above, and can allow the mevalonate pathway to function in the presence of mevalonic acid; or
(3) DNA which can hybridize with the DNA of (1) above under stringent condition, and can allow the mevalonate pathway to function in the presence of mevalonic acid.

21. Substances which specifically inhibit the non-mevalonate pathway, which are selected by the screening method of any of claims 1 to 17.

22. The substances of claim 21 which are substances having anti-bacterial activity, herbicide activity or anti-malarial activity.


SEQUENCE LISTING (15 sequences)

SEQ ID NO: 1 - DNA, 40 bp, Artificial
  An artificially synthesized primer sequence
ggaagcttggaggttgttttatgcagaaaagacaaaggga

SEQ ID NO: 2 - DNA, 28 bp, Artificial
  An artificially synthesized primer sequence
ggtctagatcactgcgcatggttgtcga

SEQ ID NO: 3 - DNA, 40 bp, Artificial
  An artificially synthesized primer sequence
ggtctagaggaggttgttttatgcgcagtgaacacccgac

SEQ ID NO: 4 - DNA, 28 bp, Artificial
  An artificially synthesized primer sequence
ggagatcttcatgcgccctcgctcagca

SEQ ID NO: 5 - DNA, 40 bp, Artificial
  An artificially synthesized primer sequence
ggagatctggaggttgttttatgacgacaggtcagcgcac

SEQ ID NO: 6 - DNA, 28 bp, Artificial
  An artificially synthesized primer sequence
ggatcgatttagatcccttccagggcag

SEQ ID NO: 7 - DNA, 40 bp, Artificial
  An artificially synthesized primer sequence
ggatcgatggaggttgttttatgaccagcgcccaacgcaa

SEQ ID NO: 8 - DNA, 28 bp, Artificial
  An artificially synthesized primer sequence
gggcatgctcatcgtgtgcttcccgtcg

SEQ ID NO: 9 - DNA, 1050 bp, Streptomyces sp.
ggaggttgttttatgcagaaaagacaaagggagctgagtgcgttgacactaccgacctcggctgagggggtatcagaaagccaccgggcccgctcggtcggcatcggtcgcgcccacgccaaggccatcctgctgggagagcatgcggtcgtctacggagcgccggcactcgctctgccgattccgcagctcacggtcacggccagcgtcggctggtcgtccgaggcctccgacagtgcgggtggcctgtcctacacgatgaccggtacgccgtcgcgggcactggtgacgcaggcctccgacggcctgcaccggctcaccgcggaattcatggcgcggatgggcgtgacgaacgcgccgcacctcgacgtgatcctggacggcgcgatcccgcacggccggggtctcggctccagcgcggccggctcacgcgcgatcgccttggccctcgccgacctcttcggccacgaactggccgagcacacggcgtacgaactggtgcagacggccgagaacatggcgcacggccgggccagcggcgtggacgcgatgacggtcggcgcgtcccggccgctgctgttccagcagggccgcaccgagcgactggccatcggctgcgacagcctgttcatcgtcgccgacagcggcgtcccgggcagcaccaaggaagcggtcgagatgctgcgggagggattcacccgcagcgccggaacacaggagcggttcgtcggccgggcgacggaactgaccgaggccgcccggcaggccctcgccgacggccggcccgaggagctgggctcgcagctgacgtactaccacgagctgctccatgaggcccgcctgagcaccgacggcatcgatgcgctggtcgaggccgcgctgaaggcaggcagcctcggagccaagatcaccggcggtggtctgggcggctgcatgatcgcacaggcccggcccgaacaggcccgggaggtcacccggcagctccacgaggccggtgccgtacagacctgggtcgtaccgctgaaagggctcgacaaccatgcgcagtga

SEQ ID NO: 10 - DNA, 1065 bp, Streptomyces sp.
ggaggttgttttatgcgcagtgaacacccgaccacgaccgtgctccagtcgcgggagcagggcagcgcggccggcgccaccgcggtcgcgcacccaaacatcgcgctgatcaagtactggggcaagcgcgacgagcggctgatcctgccctgcaccaccagcctgtcgatgacgctggacgtcttccccacgaccaccgaggtccggctcgaccccgccgccgagcacgacacggccgccctcaacggcgaggtggccacgggcgagacgctgcgccgcatcagcgccttcctctccctggtgcgggaggtggcgggcagcgaccagcgggccgtggtggacacccgcaacaccgtgcccaccggggcgggcctggcgtcctccgccagcgggttcgccgccctcgccgtcgcggccgcggccgcctacgggctcgaactcgacgaccgcgggctgtcccggctggcccgacgtggatccggctccgcctcgcggtcgatcttcggcggcttcgccgtctggcacgccggccccgacggcacggccacggaagcggacctcggctcctacgccgagccggtgcccgcggccgacctcgacccggcgctggtcatcgccgtggtcaacgccggccccaagcccgtctccagccgcgaggccatgcgccgcaccgtcgacacctcgccgctgtaccggccgtgggccgactccagtaaggacgacctggacgagatgcgctcggcgctgctgcgcggcgacctcgaggccgtgggcgagatcgcggagcgcaacgcgctcggcatgcacgccaccatgctggccgcccgccccgcggtgcggtacctgtcgccggccacggtcaccgtgctcgacagcgtgctccagctccgcaaggacggtgtcctggcctacgcgaccatggacgccggtcccaacgtgaaggtgctgtgccggcgggcggacgccgagcgggtggccgacgtcgtacgcgccgccgcgtccggcggtcaggtcctcgtcgccgggccgggagacggtgcccgcctgctgagcgagggcgcatga

SEQ ID NO: 11 - DNA, 1137 bp, Streptomyces sp.
ggaggttgttttatgacgacaggtcagcgcacgatcgtccggcacgcgccgggcaagctgttcgtcgcgggcgagtacgcggtcgtggatccgggcaacccggcgatcctggtagcggtcgaccggcacatcagcgtcaccgtgtccgacgccgacgcggacaccggggccgccgacgtcgtgatctcctccgacctcggtccgcaggcggtcggctggcgctggcacgacggccggctcgtcgtccgcgacccggacgacgggcagcaggcgcgcagcgccctggcccacgtggtgtcggcgatcgagaccgtgggccggctgctgggcgaacgcggacagaaggtccccgctctcaccctctccgtcagcagccgcctgcacgaggacggccggaagttcggcctgggctccagcggcgcggtgaccgtggcgaccgtagccgccgtcgccgcgttctgcggactcgaactgtccaccgacgaacggttccggctggccatgctcgccaccgcggaactcgaccccaagggctccggcggggacctcgccgccagcacctggggcggctggatcgcctaccaggcgcccgaccgggcctttgtgctcgacctggcccggcgcgtgggagtcgaccggacactgaaggcgccctggccggggcactcggtgcgccgactgccggcgcccaagggcctcaccctggaggtcggctggaccggagagcccgcctccaccgcgtccctggtgtccgatctgcaccgccgcacctggcggggcagcgcctcccaccagaggttcgtcgagaccacgaccgactgtgtccgctccgcggtcaccgccctggagtccggcgacgacacgagcctgctgcacgagatccgccgggcccgccaggagctggcccgcctggacgacgaggtcggcctcggcatcttcacacccaagctgacggcgctgtgcgacgccgccgaagccgtcggcggcgcggccaagccctccggggcaggcggcggcgactgcggcatcgccctgctggacgccgaggcgtcgcgggacatcacacatgtacggcaacggtgggagacagccggggtgctgcccctgcccctgactcctgccctggaagggatctaa

SEQ ID NO: 12 - DNA, 1104 bp, Streptomyces sp.
ggaggttgttttatgaccagcgcccaacgcaaggacgaccacgtacggctcgccatcgagcagcacaacgcccacagcggacgcaaccagttcgacgacgtgtcgttcgtccaccacgccctggccggcatcgaccggccggacgtgtccctggccacgtccttcgccgggatctcctggcaggtgccgatctacatcaacgcgatgaccggcggcagcgagaagaccggcctcatcaaccgggacctggccaccgccgcccgcgagaccggcgtccccatcgcgtccgggtccatgaacgcgtacatcaaggacccctcctgcgccgacacgttccgtgtgctgcgcgacgagaaccccaacgggttcgtcatcgcgaacatcaacgccaccacgacggtcgacaacgcgcagcgcgcgatcgacctgatcgaggcgaacgccctgcagatccacatcaacacggcgcaggagacgccgatgccggagggcgaccggtcgttcgcgtcctgggtcccgcagatcgagaagatcgcggcggccgtcgacatccccgtgatcgtcaaggaggtcggcaacggcctgagccggcagaccatcctgctgctcgccgacctcggcgtgcaggcggcggacgtcagcggccgcggcggcacggacttcgcccgcatcgagaacggccgccgggagctcggcgactacgcgttcctgcacggctgggggcagtccaccgccgcctgcctgctggacgcccaggacatctccctgcccgtcctcgcctccggcggtgtgcgtcacccgctcgacgtggtccgcgccctcgcgctcggcgcccgcgccgtcggctcctccgccggcttcctgcgcaccctgatggacgacggcgtcgacgcgctgatcacgaagctcacgacctggctggaccagctggcggcgctgcagaccatgctcggcgcgcgcaccccggccgacctcacccgctgcgacgtgctgctccacggcgagctgcgtgacttctgcgccgaccggggcatcgacacgcgccgcctcgcccagcgctccagctccatcgaggccctccagacgacgggaagcacacgatga

SEQ ID NO: 13 - DNA, 6798 bp, Streptomyces sp.
tacgtacttccctggcgtgtgcagcggttgacgcgccgtgccctcgctgcgagcggcgcgcacatctgacgtcctgctttattgctttctcagaactcgggacgaagcgatcccatgatcacgcgatctccatgcagaaaagacaaagggagctgagtgcgttgacactaccgacctcggctgagggggtatcagaaagccaccgggcccgctcggtcggcatcggtcgcgcccacgccaaggccatcctgctgggagagcatgcggtcgtctacggagcgccggcactcgctctgccgattccgcagctcacggtcacggccagcgtcggctggtcgtccgaggcctccgacagtgcgggtggcctgtcctacacgatgaccggtacgccgtcgcgggcactggtgacgcaggcctccgacggcctgcaccggctcaccgcggaattcatggcgcggatgggcgtgacgaacgcgccgcacctcgacgtgatcctggacggcgcgatcccgcacggccggggtctcggctccagcgcggccggctcacgcgcgatcgccttggccctcgccgacctcttcggccacgaactggccgagcacacggcgtacgaactggtgcagacggccgagaacatggcgcacggccgggccagcggcgtggacgcgatgacggtcggcgcgtcccggccgctgctgttccagcagggccgcaccgagcgactggccatcggctgcgacagcctgttcatcgtcgccgacagcggcgtcccgggcagcaccaaggaagcggtcgagatgctgcgggagggattcacccgcagcgccggaacacaggagcggttcgtcggccgggcgacggaactgaccgaggccgcccggcaggccctcgccgacggccggcccgaggagctgggctcgcagctgacgtactaccacgagctgctccatgaggcccgcctgagcaccgacggcatcgatgcgctggtcgaggccgcgctgaaggcaggcagcctcggagccaagatcaccggcggtggtctgggcggctgcatgatcgcacaggcccggcccgaacaggcccgggaggtcacccggcagctccacgaggccggtgccgtacagacctgggtcgtaccgctgaaagggctcgacaaccatgcgcagtgaacacccgaccacgaccgtgctccagtcgcgggagcagggcagcgcggccggcgccaccgcggtcgcgcacccaaacatcgcgctgatcaagtactggggcaagcgcgacgagcggctgatcctgccctgcaccaccagcctgtcgatgacgctggacgtcttccccacgaccaccgaggtccggctcgaccccgccgccgagcacgacacggccgccctcaacggcgaggtggccacgggcgagacgctgcgccgcatcagcgccttcctctccctggtgcgggaggtggcgggcagcgaccagcgggccgtggtggacacccgcaacaccgtgcccaccggggcgggcctggcgtcctccgccagcgggttcgccgccctcgccgtcgcggccgcggccgcctacgggctcgaactcgacgaccgcgggctgtcccggctggcccgacgtggatccggctccgcctcgcggtcgatcttcggcggcttcgccgtctggcacgccggccccgacggcacggccacggaagcggacctcggctcctacgccgagccggtgcccgcggccgacctcgacccggcgctggtcatcgccgtggtcaacgccggccccaagcccgtctccagccgcgaggccatgcgccgcaccgtcgacacctcgccgctgtaccggccgtgggccgactccagtaaggacgacctggacgagatgcgctcggcgctgctgcgcggcgacctcgaggccgtgggcgagatcgcggagcgcaacgcgctcggcatgcacgccaccatgctggccgcccgccccgcggtgcggtacctgtcgccggccacggtcaccgtgctcgacagcgtgctccagctccgcaaggacggtgtcctggcctacgcgaccatggacgccggtcccaacgtgaaggtgctgtgccggcgggcggacgccgagcgggtggccgacgtcgtacgcgccgccgcgtccggcggtcaggtcctcgtcgccgggccgggagacggtgcccgcctgctgagcgagggcgcatgacgacaggtcagcgcacgatcgtccggcacgcgccgggcaagctgttcgtcgcgggcgagtacgcggtcgtggatccgggcaacccggcgatcctggtagcggtcgaccggcacatcagcgtcaccgtgtccgacgccgacgcggacaccggggccgccgacgtcgtgatctcctccgacctcggtccgcaggcggtcggctggcgctggcacgacggccggctcgtcgtccgcgacccggacgacgggcagcaggcgcgcagcgccctggcccacgtggtgtcggcgatcgagaccgtgggccggctgctgggcgaacgcggacagaaggtccccgctctcaccctctccgtcagcagccgcctgcacgaggacggccggaagttcggcctgggctccagcggcgcggtgaccgtggcgaccgtagccgccgtcgccgcgttctgcggactcgaactgtccaccgacgaacggttccggctggccatgctcgccaccgcggaactcgaccccaagggctccggcggggacctcgccgccagcacctggggcggctggatcgcctaccaggcgcccgaccgggcctttgtgctcgacctggcccggcgcgtgggagtcgaccggacactgaaggcgccctggccggggcactcggtgcgccgactgccggcgcccaagggcctcaccctggaggtcggctggaccggagagcccgcctccaccgcgtccctggtgtccgatctgcaccgccgcacctggcggggcagcgcctcccaccagaggttcgtcgagaccacgaccgactgtgtccgctccgcggtcaccgccctggagtccggcgacgacacgagcctgctgcacgagatccgccgggcccgccaggagctggcccgcctggacgacgaggtcggcctcggcatcttcacacccaagctgacggcgctgtgcgacgccgccgaagccgtcggcggcgcggccaagccctccggggcaggcggcggcgactgcggcatcgccctgctggacgccgaggcgtcgcgggacatcacacatgtacggcaacggtgggagacagccggggtgctgcccctgcccctgactcctgccctggaagggatctaagaatgaccagcgcccaacgcaaggacgaccacgtacggctcgccatcgagcagcacaacgcccacagcggacgcaaccagttcgacgacgtgtcgttcgtccaccacgccctggccggcatcgaccggccggacgtgtccctggccacgtccttcgccgggatctcctggcaggtgccgatctacatcaacgcgatgaccggcggcagcgagaagaccggcctcatcaaccgggacctggccaccgccgcccgcgagaccggcgtccccatcgcgtccgggtccatgaacgcgtacatcaaggacccctcctgcgccgacacgttccgtgtgctgcgcgacgagaaccccaacgggttcgtcatcgcgaacatcaacgccaccacgacggtcgacaacgcgcagcgcgcgatcgacctgatcgaggcgaacgccctgcagatccacatcaacacggcgcaggagacgccgatgccggagggcgaccggtcgttcgcgtcctgggtcccgcagatcgagaagatcgcggcggccgtcgacatccccgtgatcgtcaaggaggtcggcaacggcctgagccggcagaccatcctgctgctcgccgacctcggcgtgcaggcggcggacgtcagcggccgcggcggcacggacttcgcccgcatcgagaacggccgccgggagctcggcgactacgcgttcctgcacggctgggggcagtccaccgccgcctgcctgctggacgcccaggacatctccctgcccgtcctcgcctccggcggtgtgcgtcacccgctcgacgtggtccgcgccctcgcgctcggcgcccgcgccgtcggctcctccgccggcttcctgcgcaccctgatggacgacggcgtcgacgcgctgatcacgaagctcacgacctggctggaccagctggcggcgctgcagaccatgctcggcgcgcgcaccccggccgacctcacccgctgcgacgtgctgctccacggcgagctgcgtgacttctgcgccgaccggggcatcgacacgcgccgcctcgcccagcgctccagctccatcgaggccctccagacgacgggaagcacacgatgacggaaacgcacgccatagccggggtcccgatgaggtgggtgggaccccttcgtatttccgggaacgtcgccgagaccgagacccaggtcccgctcgccacgtacgagtcgccgctgtggccgtcggtgggccgcggggcgaaggtctcccggctgacggagaagggcatcgtcgccaccctcgtcgacgagcggatgacccgctcggtgatcgtcgaggcgacggacgcgcagaccgcgtacatggccgcgcagaccatccacgcccgcatcgacgagctgcgcgaggtggtgcgcggctgcagccggttcgcccagctgatcaacatcaagcacgagatcaacgcgaacctgctgttcatccggttcgagttcaccaccggtgacgcctccggccacaacatggccacgctcgcctccgatgtgctcctggggcacctgctggagacgatccctggcatctcctacggctcgatctccggcaactactgcacggacaagaaggccaccgcgatcaacggcatcctcggccgcggcaagaacgtgatcaccgagctgctggtgccgcgggacgtcgtcgagaacaacctgcacaccacggctgccaagatcgtcgagctgaacatccgcaagaacctgctcggcaccctgctcgccggcggcatccgctcggccaacgcccacttcgcgaacatgctgctcggcttctacctggccaccggccaggacgccgccaacatcgtcgagggctcgcagggcgtcgtcatggccgaggaccgcgacggcgacctctacttcgcctgcaccctgccgaacctgatcgtcggcacggtcggcaacggcaagggtctcggcttcgtggagacgaacctcgcccggctcggctgccgagccgaccgcgaacccggggagaacgcccgccgcctcgccgtcatcgcggcagcgaccgtgctgtgcggtgaactctcgctgctcgcggcacagacgaacccgggcgaactcatgcgcgcgcacgtccagctggaacgcgacaacaagaccgcaaaggttggtgcatagggcatgtccatctccataggcattcacgacctgtcgttcgccacaaccgagttcgtcctgccgcacacggcgctcgccgagtacaacggcaccgagatcggcaagtaccacgtcggcatcggccagcagtcgatgagcgtgccggccgccgacgaggacatcgtgaccatggccgcgaccgcggcgcggcccatcatcgagcgcaacggcaagagccggatccgcacggtcgtgttcgccacggagtcgtcgatcgaccaggcgaaggcgggcggcgtgtacgtgcactccctgctggggctggagtcggcctgccgggtcgtcgagctgaagcaggcctgctacggggccaccgccgcccttcagttcgccatcggcctggtgcggcgcgaccccgcccagcaggtcctggtcatcgccagtgacgtctccaagtacgagctggacagccccggcgaggcgacccagggcgcggccgcggtggccatgctggtcggcgccgacccggccctgctgcgtatcgaggagccgtcgggcctgttcaccgccgacgtcatggacttctggcggcccaactacctcaccaccgctctggtcgacggccaggagtccatcaacgcctacctgcaggccgtcgagggcgcctggaaggactacgcggagcaggacggccggtcgctggaggagttcgcggcgttcgtctaccaccagccgttcacgaagatggcctacaaggcgcaccgccacctgctgaacttcaacggctacgacaccgacaaggacgccatcgagggcgccctcggccagacgacggcgtacaacaacgtcatcggcaacagctacaccgcgtcggtgtacctgggcctggccgccctgctcgaccaggcggacgacctgacgggccgttccatcggcttcctgagctacggctcgggcagcgtcgccgagttcttctcgggcaccgtcgtcgccgggtaccgcgagcgtctgcgcaccgaggcgaaccaggaggcgatcgcccggcgcaagagcgtcgactacgccacctaccgcgagctgcacgagtacacgctcccgtccgacggcggcgaccacgccaccccggtgcagaccaccggccccttccggctggccgggatcaacgaccacaagcgcatctacgaggcgcgctagcgacacccctcggcaacggggtgcgccactgttcggcgcaccccgtgccgggctttcgcacagctattcacgaccatttgaggggcgggcagccgcatgaccgacgtccgattccgcattatcggtacgggtgcctacgta

SEQ ID NO: 14 - DNA, 26 bp, Artificial
  An artificially synthesized primer sequence
ggggatccttggagcaaacgcactgc

SEQ ID NO: 15 - DNA, 27 bp, Artificial
  An artificially synthesized primer sequence
ggggatccaaaaaaggagatttgccgc